(19) United States
(12) Patent Application Publication
(10) Pub. No.: US 2025/0359788 A1
(43) Pub. Date: Nov. 27, 2025

(54) BIOSENSIGNAL ACQUISITION DEVICES, AND BIOSENSIGNAL ACQUISITION SYSTEMS AND METHODS

(71) Applicant: KARLSSONWILKER INC., Queens, NY (US)

(72) Inventors: Jan WILKER, Queens, NY (US); Yue ZHANG, Queens, NY (US)

(21) Appl. No.: 19/124,400

(22) PCT Filed: Oct. 27, 2023
(86) PCT No.: PCT/US2023/078067

§ 371 Date: Apr. 25, 2025

Related U.S. Application Data
(60) Provisional application No. 63/419,951, filed on Oct. 27, 2022.

(51) Int. Cl. A61B 5/1455; A61B 5/00; A61B 5/0531

(52) U.S. Cl. CPC A61B 5/14552; A61B 5/0531; A61B 5/6825; A61B 2560/0462; A61B 2562/0238; A61B 2562/164

(57) ABSTRACT

A biosignal acquisition system is provided that includes at least a first sensor unit and a sensor-positioning structure configured to extend across a hand of a subject. The sensor positioning structure is configured to maintain the first sensor unit at a first position on a palm of a subject. The sensor-positioning structure includes a first end configured to conform around and couple to a purlicue of a hand of the subject. The first end is C-shaped and includes a first bent section that is rigid or at least semi-rigid, the first bent section being shaped and configured to conform to and couple to the purlicue of a hand of the subject. The sensor-positioning structure further includes a second end configured to conform around and couple to a lateral portion of the hand that is opposite to the purlicue of the hand of the subject.

### CROSS-REFERENCE TO RELATED APPLICATIONS

This application claims the benefit of and priority to U.S. Provisional Patent Application Ser. No. 63/419,951 filed on Oct. 27, 2022, and entitled “Sleep Assistance Device, System, and Method,” which is expressly incorporated herein by reference.

### TECHNICAL FIELD

The present disclosure relates to the technical field of life science, in particular to a biosignal acquisition system and method, having sensors, such as an oximeter sensor or heart rate and/or galvanic skin response (GSR) sensor to determine a mood, emotional state or sleep quality or characteristics of a subject.

### BACKGROUND

Biosignal acquisitions systems are known. For example, biosignals are obtained by systems attending to determine or measure a mood, emotional state, sleep characteristics or quality, or general physiological condition of the wearer or subject. For example, a sleep assistance system determines the sleep situation of a subject, such as a human body, by collecting various biosignals, including, for example, a heart rate, an electrical skin signal, such as a galvanic skin response (GSR), etc., and then takes external intervention measures, such as controlling a room temperature, ventilation, sound, etc., to intervene in the subject's sleep. The object of such systems is to provide a system that assists people to achieve a good sleep state.

In medical institutions, special monitoring instruments are used to collect signals such as heart rate and galvanic skin response GSR, which are typically not considered to be suitable for home use. Wearable devices with such functions have appeared in the prior art. For example, Chinese patent publication CN202010829136, entitled “an intelligent bioelectric wristband and its use method”, and Chinese patent publication CN202123302512, entitled “an intelligent sports wristband integrating skin test, ultraviolet monitoring and heart rate and blood oxygen”, etc.

Further, in the market of consumer goods, devices such as Apple Watches or smart watches, purport to provide biosignals obtained from the wearer. For example, such devices purport to be able to provide data relating to sleep stages or state, mood, emotional state, or overall general physiological condition of a wearer.

### SUMMARY OF THE INVENTION

The inventors of the present application and disclosure have identified a need for a cardiac signal, such as oximeter or heart rhythm and/or GSR signal acquisition device with a high degree of integration, which is suitable for daily life. The inventors of the present application and disclosure have found that most of the biosignal collection devices are arranged at the wrist of the wearer or subject, such as bracelet structure or smart watches. However, the inventors of the present application have found that in addition to the cardiac signals, oximeter signals, the signals needed to be collected by the biosignal acquisition system, for example, in a biosignal acquisition system relating to determining a mood, sleep stages or state, emotional state, or overall general physiological condition of the wearer may also include electrical skin signals, such as a galvanic skin response (GSR), from the palm and the back of the hand, which may be collected when the wearer or subject, for example, when the wearer or subject is in a certain mood, emotional state, or sleep state. In addition to accuracy of the signal, stability and impact on human comfort are significant factors in considering the design of a device to be worn in a mood determining or assisting system or a sleep assistance system.

Further, the inventors have found that a GSR signal and an oximeter signal are particularly helpful in determining a mood, emotional state, sleep characteristics or quality, or general physiological condition of the wearer or subject. Further, the inventors of the present application and disclosure have recognized that due to the high density of sweat glands on the anterior side or palm of the hand cause a GSR signal that is obtained from the anterior side or palm of the hand to have high accuracy and more efficient detection. Further, the inventors of the present application and disclosure have found the soft tissue found in the webbing or area of the hand between the index finger and the thumb, what is known as the purlicue, is particularly effective to obtain an oximeter reading or signal from the subject.

In view of the above, the inventors of the present application and disclosure have provide a biosignal acquisition system (100) comprising at least a first sensor unit (150); and a sensor-positioning structure configured to extend across a hand (180) of a subject, the sensor positioning structure being configured to maintain the first sensor unit (150) at a first position on a palm of a hand (180) of a subject.

According to an embodiment, the sensor-positioning structure includes a first end (110), the first end (110) being configured to conform around and couple to a purlicue (181) of the hand of the subject.

According to an embodiment, the first end (110) is C-shaped and includes a first bent section (111) that is rigid or at least semi-rigid, the first bent section (111) defining a first concavity (119) and so being shaped and configured to conform to and couple to the purlicue (181) of a hand of the subject.

According to an embodiment, the sensor-positioning structure further includes a second end (120), the second end (120) being configured to conform around and couple to a lateral portion (185) of the hand that is opposite to the purlicue (181) of the hand of the subject.

According to an embodiment, the second end (120) includes a second bent (121) section that is rigid or at least semi-rigid, the second bent section (121) defining a second concavity (129) and so being shaped and configured to conform the lateral portion (185) of the hand that is opposite to the purlicue (181) of the hand of the subject.

According to an embodiment, the sensor-positioning structure further includes a connecting portion (130) extending from and coupling to the first end (110) and the second end (120).

According to an embodiment, the connecting portion (130) is configured to extend across the back of the hand of the subject.

According to an embodiment, the connecting portion (130) includes a flexible portion (135) having more flexibility than the first end (110) or the second end (120), the flexible portion (135) providing flex to the connecting portion (130).

According to an embodiment, the first sensor unit (150) includes an oximeter, the oximeter including a light emitter (155a) configured to emit light and a light detector (155b) configured to detect light emitted by the light emitter that passes through a portion of the hand of the subject, wherein the sensor-positioning structure is configured to position the light emitter at a position on the back of the hand of the subject and is configured to position the light detector at a position on the palm of the hand of the subject, or wherein the sensor-positioning structure is configured to position the light emitter at a position on the palm of the hand of the subject and is configured to position the light detector at a position on the back of the hand of the subject.

According to an embodiment, the sensor-positioning structure is configured to position the oximeter such that light emitted by the light emitter is transmitted through at least a portion of the purlicue of the hand of the subject.

According to an embodiment, the first sensor unit includes galvanic skin response (GSR) sensor.

According to an embodiment, the sensor-positioning structure is configured to position the GSR sensor at a position on the palm of the hand.

According to an embodiment, the first sensor unit (150) includes an oximeter, the oximeter including a light emitter (155a) configured to emit light and a light detector (155b) configured to detect light emitted by the light emitter that passes through a portion of the hand of the subject, the sensor-positioning structure is configured to position the light emitter (155a) at a position on the back of the hand of the subject and is configured to position the light detector (155b) at a position on the palm of the hand of the subject, the sensor-positioning structure is configured to position the oximeter such that light emitted by the light emitter is transmitted through at least a portion of the purlicue (181) of the hand of the subject, and wherein the first sensor unit includes galvanic skin response (GSR) sensor (158), wherein the sensor-positioning structure is configured to position the GSR sensor (158) at a position on the palm of the hand.

According to an embodiment, the biosignal acquisition system further comprises a control unit (140) arranged in a control housing (141), the control unit (140) being configured to receive biosignals received from the first sensor unit (150) and transmit data relating to the biosignals received from the first sensor unit (150) to a secondary device that is external to the control unit.

According to an embodiment, the control unit (140) is configured to transmit to a smart phone, laptop computer, computer, a server, or another external terminal.

According to an embodiment, the control unit (140) is configured to transmit the data relating to the biosignals received from the first sensor unit (150) to the secondary device across a network communication channel, a wireless network, or local area network.

According to an embodiment, the connecting portion (130) includes a hinge (139) that provides a pivoting point (138) such that an axis (132) of the first end (110) is arranged at an offset angle (α) from an axis (137) of the second end.

According to an embodiment, the sensor-positioning structure includes a first end (110), the first end (110) defining a first concavity (119) and so being configured to conform around and couple to a purlicue of a hand of the subject, wherein the sensor-positioning structure further includes a second end, the second end defining a second concavity (129) and so being configured to conform around and couple to a lateral portion of the hand that is opposite to the purlicue of the hand of the subject, and wherein the biosignal acquisition system further comprises a second sensor unit (160) including a second sensor (165), the sensor-positioning structure being configured to maintain the second sensor unit (165) at the second end at a second position on the palm of the subject.

According to an embodiment, the biosignal acquisition system (100) further comprises a third sensor unit (190) including a third sensor (190), wherein the sensor-positioning structure is configured to maintain the third sensor unit (195) at a position of the sensor-positioning structure facing away from a back of the hand of the subject.

A method of acquisition of data relating to a biosignal is provided, According to an example, the method comprises providing the biosignal acquisition system according to any of the above embodiments; arranging the biosignal acquisition system on a hand of the subject such that the first sensor unit is arranged and maintained at a first position on a palm of a subject; obtaining biosignals detected by the first sensor unit.

Further, in order to solve the above problems existing in the prior art, the present disclosure provides a cardiac signal, such as an oximeter or heart rate and GSR signal acquisition device for a biosignal acquisition system, which includes: a structural part and a circuit part, a main body of the circuit part is in the casing of the structural part, and the circuit part. The device includes a sensor, and a signal collection end of the sensor is on the surface of a shell of a mechanism part, and it is characterized in that the structure part includes a fixed section and a movable section.

The fixed section preferably has a “C”-shaped first shell, and the first and last ends of the first shell are hollow shells, which may be called a head-end of the shell and a tail-end of the shell.

An outer wall surface of the tail-end of the shell includes a housing facing a head-end housing is the sensor signal collection end for collecting the skin electrical signal of the palm.

An outer wall surface of the head-end housing facing the tail-end housing is a sensor signal collection end for collecting skin electrical signals, such as GSR, on the back of the hand, and a sensor signal collection end for collecting electrocardiographic signals.

A bending space of the middle part of the first shell corresponds to the shape of a corresponding side hand, for example in a preferred embodiment, the contour of the side of the hand or palm having the little finger of the palm.

The movable section is composed of an interconnected elastic stretch band and a “C”-shaped second shell. The head end of the second shell is connected to the tail end of the elastic stretch band, and the elastic stretch drives the head end to be detachably? connected to the first shell, the head end shell of the shell.

The bending space of the middle part of the second shell corresponds to the shape or contour of the side of the hand or palm of the subject having the index finger.

The tail end of the second shell is a bulging hollow shell, and the tail end shell of the first shell is also a bulging hollow shell, corresponds to the shape of the palm in the diastolic state.

By adopting the above structure, the device can be better fixed on the palm or hand of a subject, and the collection end of the corresponding sensor can be better fitted to the measurement position. And through the “C”-shaped tight palm of the movable section and the fixed section, it is stretched by elastic straps.

In a further embodiment, a material of the elastic stretchable belt is rubber, and a diamond-shaped telescopic through-hole is opened on the side of the elastic stretchable belt close to the second shell. This structure can further reduce the force of the rubber elasticity acting on the palm or hand.

In a further embodiment, the outer wall surface of the head end casing facing away from the tail end casing is provided with a solid button of the circuit part, a communication antenna, a charging connection terminal and an indicator light. With this structure, the operation of the device can be exposed to the outside of the palm without affecting the use.

In a further embodiment, the tail end casing of the first casing is bulged in the direction toward the head end casing. This structure makes the collection end of the sensor close to the palm of the hand.

By adopting the structure device, the sensor can be better positioned at the collection position, and the human body can be more comfortable in the mood detection system or system for determining a sleeping state.

### BRIEF DESCRIPTION OF THE DRAWINGS

FIG. 1A is an outline schematic diagram of a biosignal acquisition device according to a first embodiment.

FIG. 1B is a perspective view of the biosignal acquisition device according to FIG. 1A arranged on a hand of a subject.

FIG. 2A is a main perspective schematic diagram of the biosignal acquisition device of FIG. 1A.

FIG. 2B is another main perspective schematic diagram of the biosignal acquisition device of FIG. 1A arranged in another fashion.

FIG. 3A is a schematic view of a left side of the biosignal acquisition device of FIG. 2A.

FIG. 3B is a schematic view of a left side of the biosignal acquisition device of FIG. 2A.

FIG. 3C is a schematic view of a left side of another embodiment of biosignal acquisition device.

FIG. 4 is a schematic top view of a biosignal acquisition device according to another embodiment.

FIG. 5 is a perspective schematic diagram of the biosignal acquisition device of FIG. 4.

FIG. 6 is a schematic view of a side of the signal acquisition device of FIG. 4.

FIG. 7 is a schematic view of another side of the signal acquisition device of FIG. 4.

FIG. 8 shows a perspective view of a biosignal acquisition device according to another embodiment as worn on a hand of the subject.

FIG. 9 shows a different perspective view of a biosignal acquisition device according to another embodiment as worn on a hand of the subject.

FIG. 10 shows a different perspective view of a biosignal acquisition device according to another embodiment as worn on a hand of the subject.

FIG. 11 shows a different perspective view of a biosignal acquisition device according to another embodiment as worn on a hand of the subject.

### DETAILED DESCRIPTION

While the disclosure is susceptible to various modifications and alternative constructions, certain illustrative embodiments in the drawings are described below. It should be understood, however, there is no intention to limit the disclosure to the specific embodiments disclosed. On the contrary, the invention covers all modifications, alternative constructions, combinations, and equivalents falling within the spirit and scope of the disclosure.

A better understanding of the disclosure's different embodiments may be had from the following description read with the accompanying drawings in which like reference characters refer to like elements.

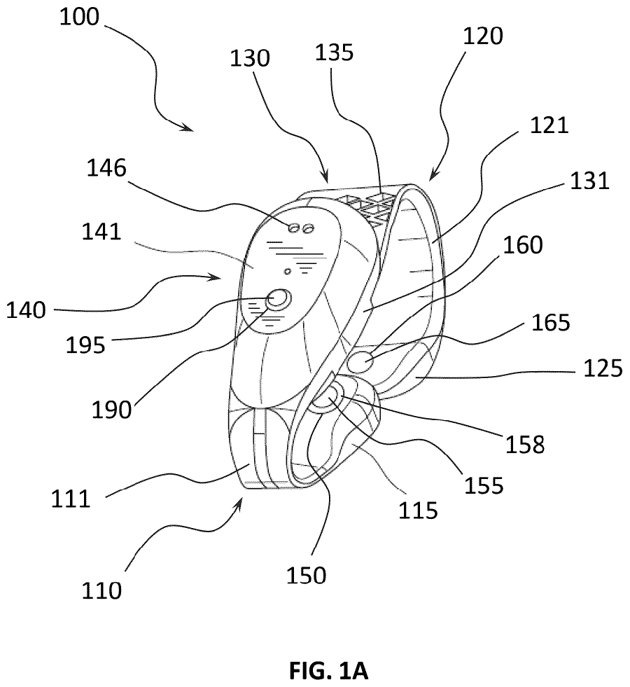

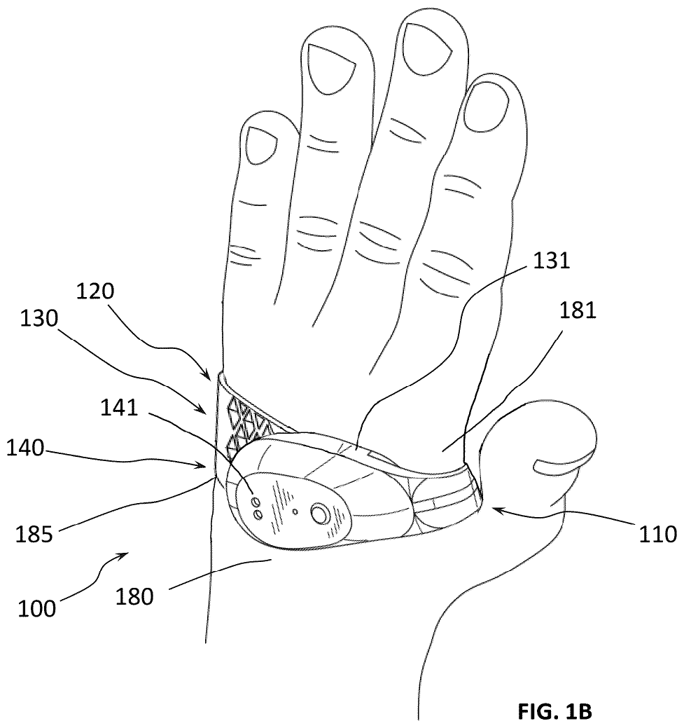

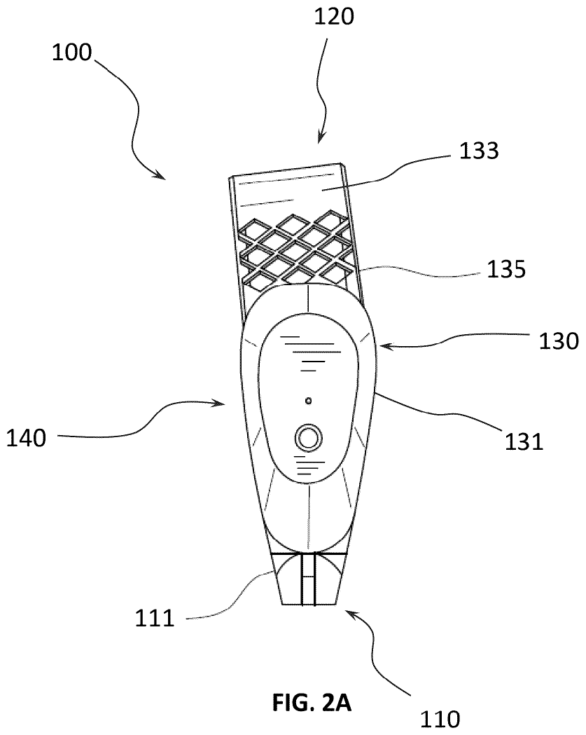

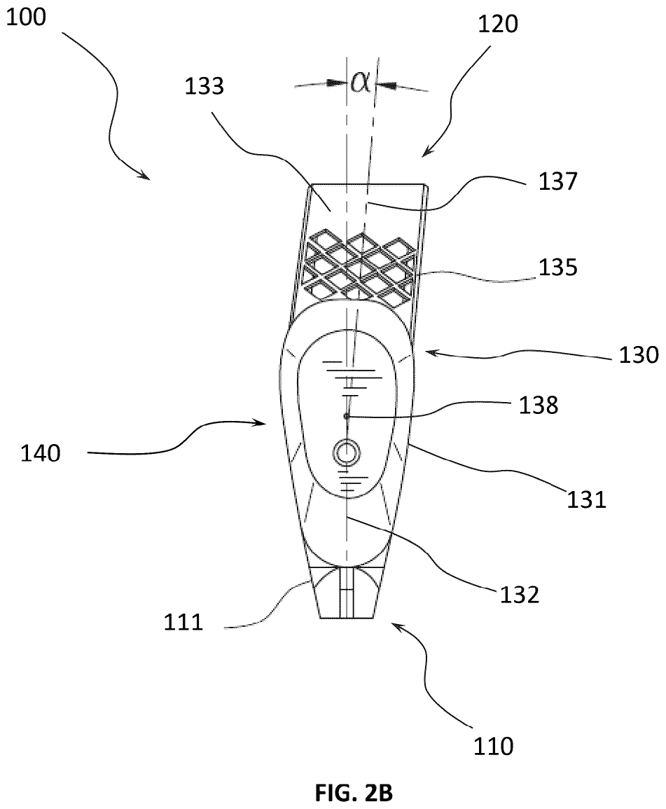

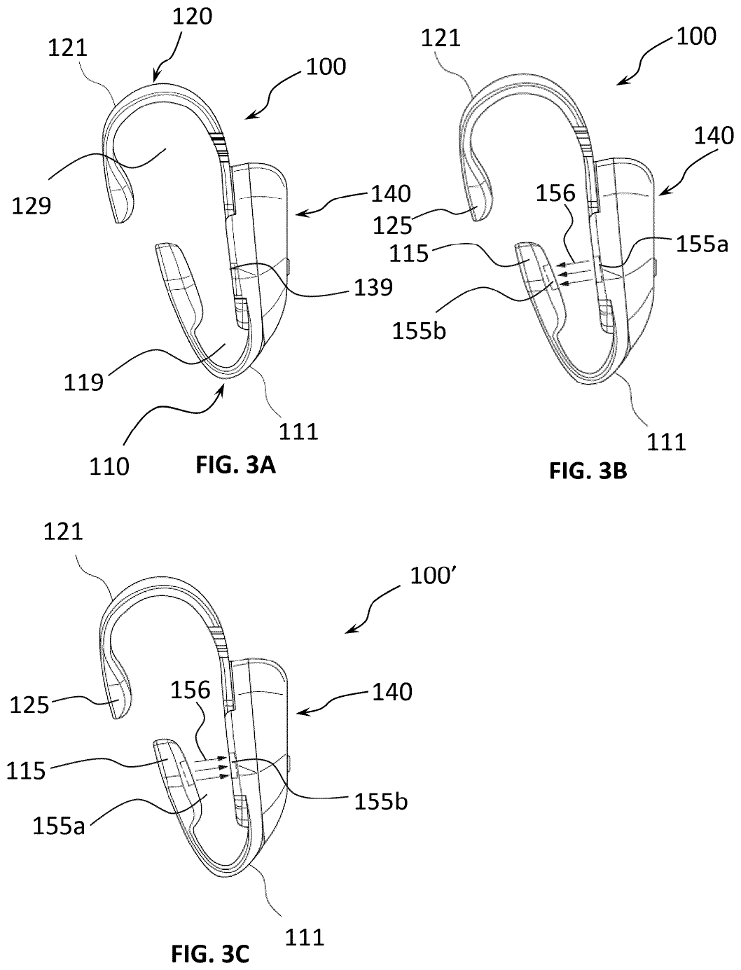

FIG. 1A shows a schematic diagram of a biosignal acquisition device according to a first embodiment. FIG. 1B shows the biosignal acquisition device of FIG. 1A as coupled or worn on the hand of a subject. FIG. 2A shows a perspective view of the device of FIG. 1A. FIG. 2B shows a perspective view of a device similar to that of FIG. 1A but arranged in a different fashion. FIGS. 3A and 3B show a side view of the biosignal acquisition device according to the embodiment of FIG. 1A.

As can be seen in FIG. 1A, in a preferred embodiment, the biosensor system 100, or what may be termed a biosignal acquisition device or a biosignal acquisition system, includes a sensor-positioning structure including a first end 110 and a second end 120, the second end 120 being arranged opposite from the first end 110. The sensor-positioning structure further includes a connecting portion 130 that connects the first end 110 to the second end 120. The first end 110 includes a first bent section 111 which is formed or arranged in a C-shape and the second end 120 includes a similar but opposing second bent section 121 that gives the second end 121 a C-shape. The first and second bent sections 111 and 121 can be generally rigid or they may be semi-rigid. As can be seen in the side view of FIG. 3A, the first bent section 111 causes the first end 110 to define a first concavity 119. And the second bent section 121 causes the second end 120 to define a second concavity 129. The first concavity is shaped to correspond in shape and size and dimensions to a first, lateral side of the hand, to the area which may be referred to as the purlicue 181, which is the soft, web of skin and soft tissue connecting between the index finger and the thumb of a user. The second concavity 129 is shaped to correspond in shape and size and dimensions to a second, lateral side 185 of the hand that is opposite to the purlicue 181. The second, lateral side 185 of the hand referring to the lateral side of the hand that is near or on the same side as the little finger.

The first end 110 of the sensor-positioning structure includes a first distal housing 115 that includes a first sensor unit 150. The first sensor unit 150 includes a first sensor 155. The first sensor unit 150 may also include second sensor 158. In a preferred embodiment, as shown in FIG. 1A, the second sensor 158 is arranged concentrically around the first sensor 155. In the embodiment of FIG. 1A, the first sensor 155 may be an oximeter including a light emitter 155a, as shown in FIG. 3B, and a light detector 155b. The oximeter is arranged such that light emitter 155a emits light 156 that transmits through the soft tissue and skin at or near the purlicue 181 and is detected by a light detector 155b. Light detector 155b may be a photodetector. Further, the light emitter 155a and the light detector 155b may be configured to emit and detect light respectively, in one or more or a plurality of different light wavelengths.

In the preferred embodiment of FIG. 3B, the light emitter is positioned by the sensor-positioning structure such that the light emitter 155a is positioned on the back of the hand, and the light detector 155b is positioned on the palm of the hand.

In an alternative embodiment, as shown in FIG. 3C, the light emitter 155A and the light detector 155b are switched such that the sensor-positioning structure positions the light emitter 155a at the palm side of the hand, and the light detector 155b is positioned on the back of the hand.

As can also be seen in FIGS. 1A and 1B, 2A, and 3A and 3B, the sensor-positioning structure and the biosignal acquisition system 100 may further include a control unit 140 The control unit 140 may include a control housing 141 configured to house the circuitry, memory, and processing devices that receive signals from the sensors of the system. The control unit 140 may further include a power source, such as a battery that is rechargeable by a physical plug in or by inductance through a powering antenna that receives power. Control unit 146 may include user input buttons or a user interface. The control unit 140 may be generally arranged on a main base 131 of the connecting portion 131. The connecting portion 131 may generally be rigid, for example, formed of a rigid plastic. Or main base 131 may be made of a semi-rigid material that would be comfortable to the wearer. The main base 131 may be connected or integrally formed with the first end 110. A connecting band 133 connects the main base 131 to the second end 120. The connecting band 133 may include a flexible portion 135. The flexible portion may be made or formed of a same material as the main base or may be made of a more flexible, elastomer or rubber-type material. The flexible portion 135 may have diamond recess or voids to provide the flexibility. Or, in place or the diamond-shaped voids, other voids may be formed therein, such as circular, triangular, square, or rectangular, or other polygon-shaped voids or recesses or designs.

The control unit 140 that is arranged in a control housing 141 may be configured to receive biosignals received from the first sensor unit 150 transmit data relating to the biosignals received from the first sensor unit 150, the control unit 140 is configured to transmit the data relating to the biosignals received from the first sensor unit 150 to the secondary device across a network communication channel, a wireless network, or local area network to a secondary device (not shown) that is external to the control unit. For example, the control unit 140 may be configured to transmit to a smart phone, laptop computer, computer, a server, or another external terminal. The control unit 140 may be configured to receive the data or biosignals from the first sensor unit 150, the second sensor unit 160, and/or the third sensor unit 190, or any other sensor of input of the biosignal acquisition system through either a hardwired connection or by a wireless connection. The control unit 140 may be configured to transmit the data relating to the biosignals received from the first sensor unit 150, the second sensor unit 160, or the third sensor unit 190 to the secondary device across a network communication channel, a wireless network, or local area network.

Further, the data obtained from the sensors may be transmitted directly by the control unit 140 through a network to a remote server, where the data from the sensors may be processed and analyzed, and results may be transmitted from the remote server to the user or to another device of the user to be viewed, such as a phone, tablet, computer, or laptop, or some other terminal.

Further, the control unit 140 itself may include a processor configured to perform analysis of the signal received from any of the sensors of the biosignal acquisition system. The control unit 140 may then transmit or output unprocessed data relating to the biosignals, partially processed data obtained from the received biosignal, or fully processed data and analysis data based on the received biosignals.

At the distal end of the second end 120, a second end distal housing 125 may be provided. The second end distal housing 125 may provide a housing for a second sensor unit 160, which may include a second sensor 165. The second sensor 165 may be a GSR sensor that functions in cooperation with the second sensor 158 of the first sensor unit 155. Accordingly, the sensor-positioning structure is arranged to provide corresponding sensor or electrodes 156 and 165 to provide a GSR sensor on the palm or anterior surface of the hand of the subject.

Further, a third sensor unit 190 may be provided on a side of the control unit 140 that is opposite from the user's hand. The third sensor unit 190 may include a third sensor 195, which may, for example, be an electrode that may easily be coupled to the head, scalp, or forehead of the user when the user is in a reclined, relaxed, or laying-down position The electrode of the third sensor 195 may serve as an electrocardiogram (ECG or EKG) signal or even to receive electroencephalogram (EEG) signals.

The biosignal acquisition system of the embodiment of FIG. 2A is arranged in a first configuration, which lends itself to be worn comfortably by the user on the right hand. By comparison, as can be seen in FIG. 2B, the same or a similar biosignal acquisition system may be arranged in a second configuration wherein an axis 132 of the main base 131 at an offset angle a from an axis 137 of the connecting band 133. This change in angle between the main base 131 and first end 110 and the connecting band 133 is due to a pivoting at pivot point 138 due to hinge 139, with the hinging include, for example, a pin and a corresponding pivot hole through which the pin enters or is mated.

Galvanic skin response (GSR) is based on a the measured skin resistance due to the sweat glands of the skin. Sweating is controlled by the sympathetic nervous system, and skin conductance is an indication of psychological or physiological arousal. It is understood that if the sympathetic branch of the autonomic nervous system becomes aroused, sweat gland activity increases, which in turn increases skin conductance. In this way, skin conductance can be a measure of emotional and sympathetic responses, and reduced skin conductance is correlated with a relaxation of the subject.

In addition to the above-noted sensors, other various biosensors may be included with the biosignal acquisition system as described herein. It is noted that the biosensors should not be limited to the specific sensors described herein, but rather the significance of the sensor is to obtain biosignal data related to the desired adjustment in mood, emotions, feelings, or affective state of the subject. Such sensors therefore may include, but are not limited to, a sensor that obtains one or more biosignals obtained from the subject include data relating to electrodermal activity (EDA), galvanic skin response (GSR), electrodermal response (EDR), psychogalvanic reflex (PGR), skin conductance response (SCR), sympathetic skin response (SSR) and skin conductance level (SCL), blood pressure (BP), pulse oximetry, oxygen saturation, electroencephalography (EEG), electromyography (EMG), body movement based on one or more accelerometers or one or more gyroscopes, electrocardiography (ECG), temperature of the subject, thermal imaging, respiration, visual images of the subject, heart rate (HR), heart rate variability (HRV), photoelectric plethysmography (PPG), photoplethysmography imaging (PPGI), prefrontal cortex activity, oxyhemoglobin (oxy-Hb) concentration, cortisol levels including salivary cortisol levels, hair cortisol levels, and/or fingernail cortisol levels, pupil dilation, pupillometry, pulsimetry, accelerated plethysmography (APG), optical imaging of tissues of the subject including functional near infrared spectroscopy (fNIRS), functional magnetic resonance imaging (fMRI), computed tomography (CT), magnetoencephalography (MEG), positron emission tomography (PET), or infrared spectroscopy (NIRS).

The biosignal acquisition system as described herein lends itself particularly well to a mood-adjusting method and system, as described in detail in WO 2022/109007 A1, as published on May 27, 2022, by the same applicant as the present application. WO 2022/109007 A1 is incorporated herein by reference. Or the biosignal acquisition system as described herein may be used in many other applications or systems where the mood, emotional state, sleep state, or general physiological state of the subject is to be determined. These may include sleep or mood enhancement systems and methods, focus groups studies, such as in obtaining data relating to users' or consumers' emotional responses to products or services, or to obtain data relating to users' or viewers' reaction to viewing media content. But of particular significance, the biosignal acquisition systems as described herein provide particularly useful and efficiently and comfortably obtained and accurate readings for an oximetry sensor, placed at the purlicue of the subject and GSR readings measured from the palm of the hand or anterior surface of the hand. By comparison, often oximeter devices are used on the ends of the fingers of the subject, but finger bones impede the transmission of light through the flesh of the finger at this location. Similarly, oximetry readings may by obtained at the subject's earlobes. But this location does not lend itself to comfortably obtaining such signals.

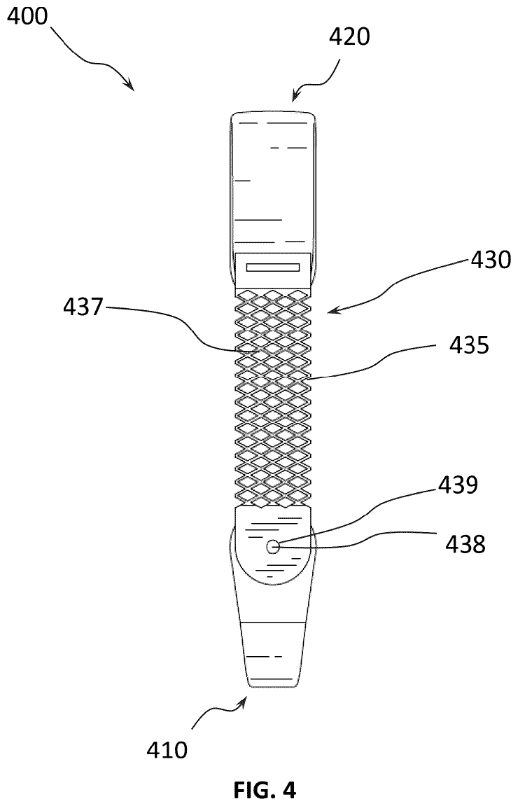

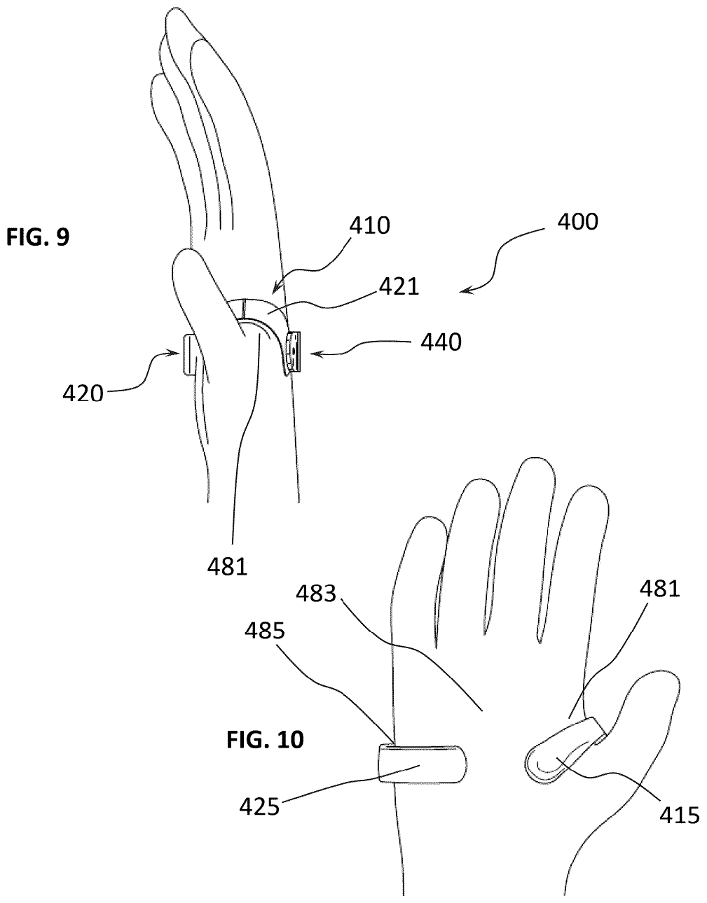

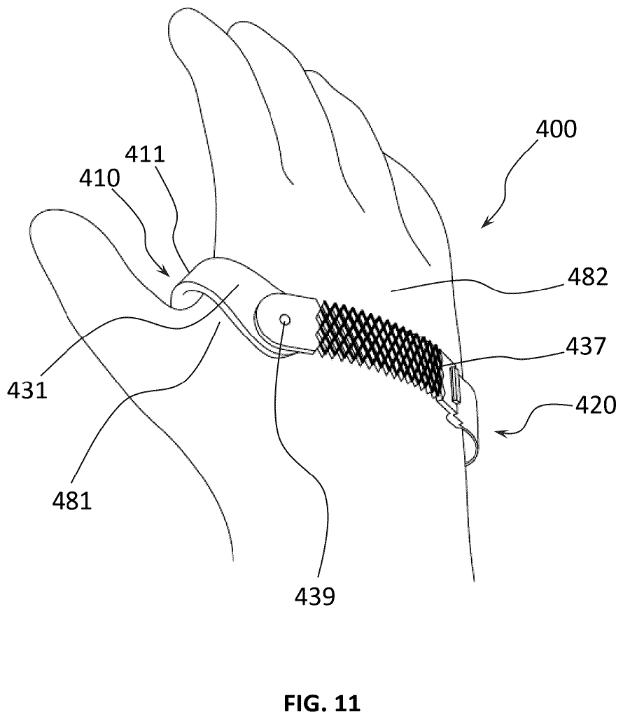

FIGS. 4-6 show a schematic diagram of a biosignal acquisition device or system 400 according to a second embodiment. The second embodiment of FIGS. 4-6 may be preferable due to having generally a reduced profile, which may be more comfortable for the user. And FIGS. 8-11 show the biosignal acquisition device of FIGS. 4-6 as coupled or worn on the hand of a subject.

As can be seen in FIG. 4, in another preferred embodiment, the biosensor system 400, or what may be termed a biosignal acquisition device or a biosignal acquisition system, includes a sensor-positioning structure including a first end 410 and a second end 420, the second end 420 being arranged opposite from the first end 410. The sensor-positioning structure further includes a connecting portion 430 that connects the first end 410 to the second end 420. The first end 410 includes a first bent section 411 and the second end 420 includes an opposing second bent section 421. As can be seen in the side view of FIG. 6, the first bent section 411 causes the first end 410 to define a first concavity 419. And the second bent section 421 causes the second end 420 to define a second concavity 429. The first concavity 419 is shaped to correspond in shape and size and dimensions to a first, lateral side of the hand, to the area which may be referred to as the purlicue 481, which is the soft, web of skin and soft tissue connecting between the index finger and the thumb of a user. The second concavity 429 is shaped to correspond in shape and size and dimensions to a second, lateral side 485 of the hand that is opposite to the purlicue 481. The second, lateral side 485 of the hand referring to the lateral side of the hand that is near or on the same side as the little finger.

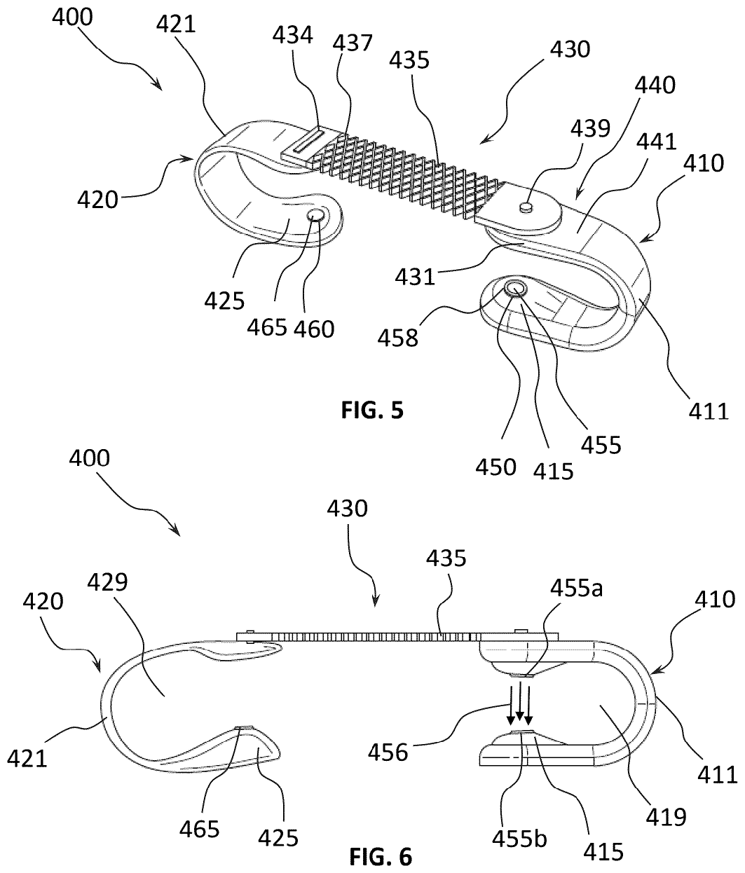

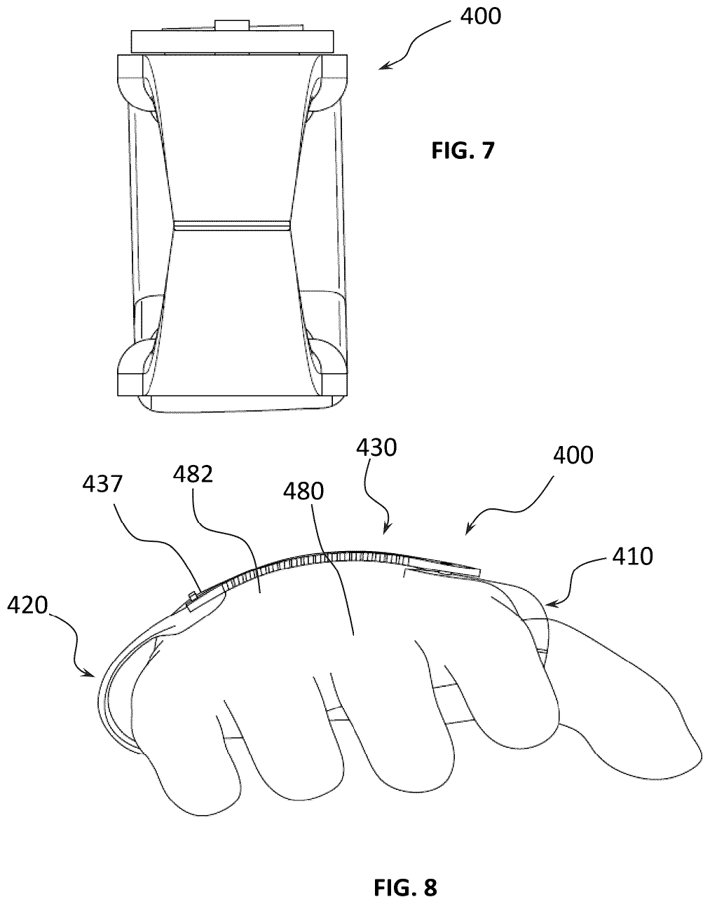

In the embodiment of FIGS. 4-6, the first end 410 of the sensor-positioning structure includes a first distal housing 415 that includes a first sensor unit 450. The first sensor unit 450 includes a first sensor 455. The first sensor unit 450 may also include a second sensor 458. In a preferred embodiment, the second sensor 458 is arranged concentrically around the first sensor 455. In the embodiment of FIG. 5, the first sensor 455 may by an oximeter including a light emitter 455a, as shown in FIG. 6, and a light detector 455b. The oximeter is arranged such that light emitter 455a emits light 456 that transmits through the soft tissue and skin at or near the purlicue 481 and is detected by a light detector 455b. Light detector 455b may be a photodetector. Further, the light emitter 455a and the light detector 455b may be configured to emit and detect light respectively, in one or more or a plurality of different light wavelengths.

In the preferred embodiment of FIG. 6, the light emitter is positioned by the sensor-positioning structure such that the light emitter 455a is positioned on the back of the hand, and the light detector 455b is positioned on the palm of the hand.

Of course, in an alternative embodiment, the light emitter 455A and the light detector 455b may be switched such that the sensor-positioning structure positions the light emitter 455a at the palm side of the hand, and the light detector 455b is positioned on the back of the hand.

As can also be seen in FIGS. 4-6 and 8-11, the sensor-positioning structure and the biosignal acquisition system 400 may further include a control unit 440 or a reduced profile. The control unit 440 may include a control housing 441 configured to house the circuitry, memory, and processing devices that receive signals from the sensors of the system. The control unit 440 may further include a power source, such as a battery that is rechargeable by a physical plug in or by inductance through a powering antenna that receives power. Control unit 446 may include user input buttons or a user interface. The control unit 440 may be generally arranged on a main base 431 of the connecting portion 431. The connecting portion 431 may generally be rigid, for example, formed of a rigid plastic. Or main base 431 may be made of a semi-rigid material that would be comfortable to the wearer. The main base 131 may be connected or integrally formed with the first end 410. A connecting band 133 connects the main base 431 to the second end 120. The connecting band 133 may include a flexible portion 435. The flexible portion may be made or formed of a same material as the main base or may be made of a more flexible, elastomer or rubber-type material. The flexible portion 435 may have diamond recess or voids to provide the flexibility. Or, in place or the diamond-shaped voids, other voids may be formed therein, such as circular, triangular, square, or rectangular, or other polygon-shaped voids or recesses or designs. The control unit 440 may be configured to receive the data or biosignals from the first sensor unit 450, the second sensor unit 460, and/or a third or any other sensor of input of the biosignal acquisition system 400 through either a hardwired connection or by a wireless connection.

At the distal end of the second end 420, a second end distal housing 425 may be provided. The second end distal housing 425 may provide a housing for a second sensor unit 460, which may include a second sensor 465. The second sensor 465 may be a GSR sensor that functions in cooperation with the second sensor 458 of the first sensor unit 455. Accordingly, the sensor-positioning structure is arranged to provide corresponding sensor or electrodes 456 and 465 to provide a GSR sensor on the palm or anterior surface of the hand of the subject.

The biosignal acquisition system of the embodiment of FIG. 4-6 may be arranged in a left-handed or right-handed configuration, due to a change in angle between the main base 431 and first end 410 and the connecting band 433 is due to a pivoting at pivot point 438 due to hinge 439, with the hinge include, for example, a pin and a corresponding pivot hole through which the pin enters or is mated.

Thus, in the embodiment of FIGS. 1A and 1B or FIGS. 4 6, the first end 110, 410, control portions 140, 440 and the main base plate 131, 431 may generally be considered a “fixed section”, whereas the connecting band 133, 433, with the flexible portion 135, 435 may be considered as a movable section. The connecting band 133, 433 of each embodiment may also be a flexible or elastic belt.

As shown, cardiac signal, such as an oximeter or a heart rate and GSR signal acquisition device may be provided in the device, to provide biosignals, which may be used, for example in a biosignal acquisition system is provided, which comprises: a structural part and a circuit part, the main body of the circuit part is in the casing of the structural part, and the sensor signal acquisition end of the circuit part is on the surface of the casing of the mechanism part is that the structural part includes a fixed segment and a movable segment.

The fixed section is a “C”-shaped first shell, and the first and last ends of the first shell are hollow shells, which are called the head-end shell and the tail-end shell.

The outer wall surface of the tail-end housing facing the head-end housing is the sensor signal collection end for collecting the skin electrical signal of the palm.

The outer wall surface of the head-end housing facing the tail-end housing is a sensor signal collection end for collecting skin electrical signals on the back of the hand, and a sensor signal collection end for collecting electrocardiographic signals.

The bending space of the middle part of the first shell corresponds to the shape of the corresponding side of the little finger of the palm;

The movable section is composed of an interconnected elastic stretch band and a “C”-shaped second shell, the head end of the second shell is connected to the tail end of the elastic stretch band, and the elastic stretch drives the head end to be detachably connected to the first shell, the head end shell of the shell.

The bending space of the middle part of the second shell corresponds to the shape of the corresponding side of the index finger of the palm.

The tail end of the second shell is a bulging hollow shell, and the tail end shell of the first shell is also a bulging hollow shell, corresponding to the shape of the palm in the diastolic state.

By adopting the above structure, the device can be better fixed on the palm of a person, and the collection end of the corresponding sensor can be better fitted to the measurement position. And through the “C”-shaped tight palm of the movable section and the fixed section, it is stretched by elastic straps.

In this example, the elastic belt is made of rubber material, and a diamond-shaped elastic through hole is opened on the side of the elastic belt close to the second shell. This structure can further reduce the force of the rubber elasticity acting on the palm.

On the outer wall surface of the head-end housing facing away from the tail-end housing, there are solid buttons of the circuit part, a communication antenna, a charging connection terminal and an indicator light. With this structure, the operation of the device can be exposed to the outside of the palm without affecting the use.

The rear end casing of the first casing is bulged in the direction towards the head end casing. This structure makes the collection end of the sensor close to the palm of the hand.

Embodiments of the present disclosure may comprise or utilize a special-purpose or general-purpose computer system that includes computer hardware, such as, for example, one or more processors and system memory, as discussed in greater detail below. Embodiments within the scope of the present disclosure also include physical and other computer-readable media for carrying or storing computer-executable instructions and/or data structures. Such computer-readable media can be any available media that can be accessed by a general-purpose or special-purpose computer system. Computer-readable media that store computer-executable instructions and/or data structures are computer storage media. Computer-readable media that carry computer-executable instructions and/or data structures are transmission media. Thus, by way of example, embodiments of the disclosure can comprise at least two distinctly different kinds of computer-readable media: computer storage media and transmission media.

Computer storage media are physical storage media that store computer-executable instructions and/or data structures. Physical storage media include computer hardware, such as RAM, ROM, EEPROM, solid state drives (“SSDs”), flash memory, phase-change memory (“PCM”), optical disk storage, magnetic disk storage or other magnetic storage devices, or any other hardware storage device(s) which can be used to store program code in the form of computer-executable instructions or data structures, which can be accessed and executed by a general-purpose or special-purpose computer system to implement the disclosed functionality of the disclosure.

Transmission media can include a network and/or data links which can be used to carry program code in the form of computer-executable instructions or data structures, and which can be accessed by a general-purpose or special-purpose computer system. A “network” may be defined as one or more data links that enable the transport of electronic data between computer systems and/or modules and/or other electronic devices. When information is transferred or provided over a network or another communications connection (either hardwired, wireless, or a combination of hardwired or wireless) to a computer system, the computer system may view the connection as transmission media. Combinations of the above should also be included within the scope of computer-readable media.

Further, upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module (e.g., a “NIC”), and then eventually transferred to computer system RAM and/or to less volatile computer storage media at a computer system. Thus, it should be understood that computer storage media can be included in computer system components that also (or even primarily) utilize transmission media.

Computer-executable instructions may comprise, for example, instructions and data which, when executed by one or more processors, cause a general-purpose computer system, special-purpose computer system, or special-purpose processing device to perform a certain function or group of functions. Computer-executable instructions may be, for example, binaries, intermediate format instructions such as assembly language, or even source code.

The disclosure of the present application may be practiced in network computing environments with many types of computer system configurations, including, but not limited to, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. As such, in a distributed system environment, a computer system may include a plurality of constituent computer systems. In a distributed system environment, program modules may be located in both local and remote memory storage devices.

The disclosure of the present application may also be practiced in a cloud-computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

Some embodiments, such as a cloud-computing environment, may comprise a system that includes one or more hosts that are each capable of running one or more virtual machines. During operation, virtual machines emulate an operational computing system, supporting an operating system and perhaps one or more other applications as well. In some embodiments, each host includes a hypervisor that emulates virtual resources for the virtual machines using physical resources that are abstracted from view of the virtual machines. The hypervisor also provides proper isolation between the virtual machines. Thus, from the perspective of any given virtual machine, the hypervisor provides the illusion that the virtual machine is interfacing with a physical resource, even though the virtual machine only interfaces with the appearance (e.g., a virtual resource) of a physical resource. Examples of physical resources including processing capacity, memory, disk space, network bandwidth, media drives, and so forth.

Certain terms are used throughout the description and claims to refer to particular methods, features, or components. As those having ordinary skill in the art will appreciate, different persons may refer to the same methods, features, or components by different names. This disclosure does not intend to distinguish between methods, features, or components that differ in name but not function. The figures are not necessarily drawn to scale. Certain features and components herein may be shown in exaggerated scale or in somewhat schematic form and some details of conventional elements may not be shown or described in interest of clarity and conciseness.

Although various example embodiments have been described in detail herein, those skilled in the art will readily appreciate in view of the present disclosure that many modifications are possible in the example embodiments without materially departing from the concepts of present disclosure. Accordingly, any such modifications are intended to be included in the scope of this disclosure. Likewise, while the disclosure herein contains many specifics, these specifics should not be construed as limiting the scope of the disclosure or of any of the appended claims, but merely as providing information pertinent to one or more specific embodiments that may fall within the scope of the disclosure and the appended claims. Any described features from the various embodiments disclosed may be employed in combination. In addition, other embodiments of the present disclosure may also be devised which lie within the scopes of the disclosure and the appended claims. Each addition, deletion, and modification to the embodiments that falls within the meaning and scope of the claims is to be embraced by the claims.

Certain embodiments and features may have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges may appear in one or more claims below. Any numerical value is “about” or “approximately” the indicated value, and takes into account experimental error and variations that would be expected by a person having ordinary skill in the art.

### Combinability of Embodiments and Features

This disclosure provides various examples, embodiments, features, and methods of a biosensor devices, biosensor systems, and methods, which improve detection of biosignals of a patient. Unless expressly stated, or unless such examples, embodiments, and features would be mutually exclusive, the various examples, embodiments, features, and methods disclosed herein should be understood to be combinable with other examples, embodiments, features, or methods described herein.

In addition to the above, further embodiments and examples include the following enumerated embodiments and methods.

A biosignal acquisition system (100) comprising: at least a first sensor unit (150); and a sensor-positioning structure configured to extend across a hand (180) of a subject, the sensor positioning structure being configured to maintain the first sensor unit (150) at a first position on a palm of a hand (180) of a subject.
2. The biosignal acquisition system (100) according to any one or a combination of one or more of 1 above and 3-19 below, wherein the sensor-positioning structure includes a first end (110), the first end (110) being configured to conform around and couple to a purlicue (181) of the hand of the subject.
3. The biosignal acquisition system (100) according to any one or a combination of one or more of 1-2 above and 4-19 below, wherein the first end (110) is C-shaped and includes a first bent section (111) that is rigid or at least semi-rigid, the first bent section (111) defining a first concavity (119) and so being shaped and configured to conform to and couple to the purlicue (181) of a hand of the subject.
4. The biosignal acquisition system (100) according to any one or a combination of one or more of 1-3 above and 5-19 below, wherein the sensor-positioning structure further includes a second end (120), the second end (120) being configured to conform around and couple to a lateral portion (185) of the hand that is opposite to the purlicue (181) of the hand of the subject.
5. The biosignal acquisition system (100) according to any one or a combination of one or more of 1-4 above and 6-19 below, wherein the second end (120) includes a second bent (121) section that is rigid or at least semi-rigid, the second bent section (121) defining a second concavity (129) and so being shaped and configured to conform the lateral portion (185) of the hand that is opposite to the purlicue (181) of the hand of the subject.
6. The biosignal acquisition system (100) according to any one or a combination of one or more of 1-5 above and 7-19 below, wherein the sensor-positioning structure further includes a connecting portion (130) extending from and coupling to the first end (110) and the second end (120).
7. The biosignal acquisition system (100) according to claim 6, wherein the connecting portion (130) is configured to extend across the back of the hand of the subject.
8. The biosignal acquisition system (100) according to any one or a combination of one or more of 1-7 above and 9-19 below, wherein the connecting portion (130) includes a flexible portion (135) having more flexibility than the first end (110) or the second end (120), the flexible portion (135) providing flex to the connecting portion (130).
9. The biosignal acquisition system (100) according to any one or a combination of one or more of 1-8 above and 10-19 below, wherein the first sensor unit (150) includes an oximeter, the oximeter including a light emitter (155a) configured to emit light and a light detector (155b) configured to detect light emitted by the light emitter that passes through a portion of the hand of the subject, wherein the sensor-positioning structure is configured to position the light emitter at a position on the back of the hand of the subject and is configured to position the light detector at a position on the palm of the hand of the subject, or wherein the sensor-positioning structure is configured to position the light emitter at a position on the palm of the hand of the subject and is configured to position the light detector at a position on the back of the hand of the subject.
10. The biosignal acquisition system (100) according to any one or a combination of one or more of 1-9 above and 11-19 below, wherein the sensor-positioning structure is configured to position the oximeter such that light emitted by the light emitter is transmitted through at least a portion of the purlicue of the hand of the subject.
11. The biosignal acquisition system (100) according to any one or a combination of one or more of 1-10 above and 12-19 below, wherein the first sensor unit includes galvanic skin response (GSR) sensor.
12. The biosignal acquisition system (100) according to any one or a combination of one or more of 1-11 above and 13-19 below, wherein the sensor-positioning structure is configured to position the GSR sensor at a position on the palm of the hand.
13. The biosignal acquisition system (100) according to any one or a combination of one or more of 1-12 above and 14-19 below, wherein the first sensor unit (150) includes an oximeter, the oximeter including a light emitter (155a) configured to emit light and a light detector (155b) configured to detect light emitted by the light emitter that passes through a portion of the hand of the subject, wherein the sensor-positioning structure is configured to position the light emitter (155a) at a position on the back of the hand of the subject and is configured to position the light detector (155b) at a position on the palm of the hand of the subject, wherein the sensor-positioning structure is configured to position the oximeter such that light emitted by the light emitter is transmitted through at least a portion of the purlicue (181) of the hand of the subject, and wherein the first sensor unit includes galvanic skin response (GSR) sensor (158), wherein the sensor-positioning structure is configured to position the GSR sensor (158) at a position on the palm of the hand.
14. The biosignal acquisition system (100) according to any one or a combination of one or more of 1-3 above and 15-19 below, wherein the biosignal acquisition system further comprises a control unit (140) arranged in a control housing (141), the control unit (140) being configured to receive biosignals received from the first sensor unit (150) and transmit data relating to the biosignals received from the first sensor unit (150) to a secondary device that is external to the control unit.
15. The biosignal acquisition system (100) according to any one or a combination of one or more of 1-4 above and 16-19 below, wherein the control unit (140) is configured to transmit to a smart phone, laptop computer, computer, a server, or another external terminal.
16. The biosignal acquisition system (100) according to any one or a combination of one or more of 1-15 above and 17-19 below, wherein the control unit (140) is configured to transmit the data relating to the biosignals received from the first sensor unit (150) to the secondary device across a network communication channel, a wireless network, or local area network.
17. The biosignal acquisition system (100) according to any one or a combination of one or more of 1-16 above and 18-19 below, wherein the connecting portion (130) includes a hinge (139) that provides a pivoting point (138) such that an axis (132) of the first end (110) is arranged at an offset angle (α) from an axis (137) of the second end.
18. The biosignal acquisition system (100) according to any one or a combination of one or more of 1-17 above and 19 below, wherein the sensor-positioning structure includes a first end (110), the first end (110) defining a first concavity (119) and so being configured to conform around and couple to a purlicue of a hand of the subject, wherein the sensor-positioning structure further includes a second end, the second end defining a second concavity (129) and so being configured to conform around and couple to a lateral portion of the hand that is opposite to the purlicue of the hand of the subject, and wherein the biosignal acquisition system further comprises a second sensor unit (160) including a second sensor (165), the sensor-positioning structure being configured to maintain the second sensor unit (165) at the second end at a second position on the palm of the subject.
19. The biosignal acquisition system (100) according to any one or a combination of one or more of 1-18 above, further comprising a third sensor unit (190) including a third sensor (190), wherein the sensor-positioning structure is configured to maintain the third sensor unit (195) at a position of the sensor-positioning structure facing away from a back of the hand of the subject.
20. A method of data relating to a biosignal, the method comprising: providing the biosignal acquisition system according to any of 1-19 above; arranging the biosignal acquisition system on a hand of the subject such that the first sensor unit is arranged and maintained at a first position on a palm of a subject; obtaining biosignals detected by the first sensor unit.

In addition to the above, further embodiments and examples include the following enumerated embodiments and methods.
1. A biosignal acquisition device comprising: a heart rate and a GSR signal acquisition device for a biosignal acquisition system, which may be a mood determining system or a sleep state determining system; a structural part; a circuit part, wherein a main body of the circuit part is in the casing of the structural part, and a sensor signal acquisition end of the circuit part is on a surface of a casing of a mechanism part; wherein the structural part includes a fixed section and a movable section; wherein the fixed section is a “C”-shaped first shell, having a first end and a second end which are hollow shells, which are called the head-end shell and the tail-end shell; an outer wall surface of a tail-end housing facing a head-end housing houses the sensor signal collection end for collecting the skin electrical signal of the palm; an outer wall surface of the head-end housing facing the tail-end housing houses a sensor signal collection end for collecting skin electrical signals on the back of the hand, and a sensor signal collection end for collecting electrocardiographic signals; a bending space of the middle part of the first shell corresponds to the shape of the corresponding side hand or palm having the little finger; a movable section is composed of an interconnected elastic stretch band and a “C”-shaped second shell, the head-end of the second shell is connected to the tail-end of the elastic stretch band, and the elastic stretch drives the head-end to be detachably connected to the first shell, the head-end shell of the shell; the bending space of the middle part of the second shell corresponds to the shape of the corresponding side of the hand having the index finger; the tail end of the second shell is a bulging hollow shell, and the tail end shell of the first shell is also a bulging hollow shell, which corresponds to the shape of the palm in the diastolic state.
2. The biosignal acquisition device according to one or more or a combination of 1 above or 3-8 below, wherein the elastic band is made of rubber, and/or a diamond-shaped through hole is opened on the side of the elastic band close to the second shell.
3. The biosignal acquisition device according to one or more or a combination of 1-2 above or 4-8 below, wherein the outer wall surface of the head-end housing facing away from the tail-end housing has a solid button of the circuit part, a communication antenna, and/or a charging connection terminal and indicator lights.
4. The biosignal acquisition device according to one or more or a combination of 1-3 above or 5-8 below, wherein the rear end casing of the first casing is bulged in the direction toward the head-end casing.
5. The biosignal acquisition device according to one or more or a combination of 1-4 above or 6-8 below, wherein data obtained by the biosignal acquisition device is transmitted to the remote server through Bluetooth and Wi-Fi, and optionally where the data serves for remote treatment and provide real-time data feedback for remote medical diagnosis and treatment.
6. The biosignal acquisition device according to one or more or a combination of 1-5 above or 7-8 below, wherein the biosignal acquisition device is configured to be connected to monitoring edge devices to provide reference for medical clinical trials; and optionally wherein due to the real-time, objectivity, and/or authenticity of data, the biosignal acquisition device is configured to be used as first-hand objective clinical monitoring data.
7. The biosignal acquisition device according to one or more or a combination of 1-6 above or 8 below, wherein the biosignal acquisition device is configured to monitor the subject's by obtaining monitored data; and optionally wherein the monitored data is used as an auxiliary reference for the subconscious observation and measurement.
8. The biosignal acquisition device according to one or more or a combination of 1-6 above or 8 below, wherein the biosignal acquisition device is configured to be tested when the subject is awake, as it is a real-time test of heart rate and GSR data, and these data assist in inferring the stress level and relaxation level; and optionally wherein the biosignal acquisition device is configured to be used by game players as an add-on to a gameplay dimension, which will change parameters of the game being played.
9. A biosignal acquisition system for a mood monitoring or sleep monitoring or assistance system comprising: a biosignal acquisition device according to one or more or a combination of 1-8 above; a data receiving device configured to receive data transmitted from the biosignal acquisition device, the data receiving device having a memory to store the data transmitted from the biosignal acquisition device; and a processing and controlling device configured to determine a mood or quality or state of sleep of a subject based on the received data transmitted from the biosignal acquisition device and take steps to adjust the mood or quality or state of the sleep of the subject.
10. A method of acquiring a biosignal, the method comprising: providing a biosignal acquisition device according to one or more or a combination of 1-8 above; obtaining a biosignal from a subject with biosignal acquisition device, the biosignal from the subject being indicative of the mood or quality or state of sleep of the subject.
11. A biosignal acquisition device comprises: one or more biosignal sensors; a structural part; a circuit part, wherein the biosignal acquisition device satisfies one or more of the following characteristics (a) to (i): (a) a main body of the circuit part is in the casing of the structural part, and a sensor signal acquisition end of the circuit part is on a surface of a casing of a mechanism part; (b) wherein the structural part includes a fixed section and a movable section; (c) wherein the fixed section is a “C”-shaped first shell, having a first end and a second end which are hollow shells, which are called the head-end shell and the tail-end shell; (d) an outer wall surface of a tail-end housing facing a head-end housing houses the sensor signal collection end for collecting the skin electrical signal of the palm; (e) an outer wall surface of the head-end housing facing the tail-end housing houses a sensor signal collection end for collecting skin electrical signals on the back of the hand, and a sensor signal collection end for collecting electrocardiographic signals; (f) a bending space of the middle part of the first shell corresponds to the shape of the corresponding side hand or palm having the little finger; (g) a movable section is composed of an interconnected elastic stretch band and a “C”-shaped second shell, the head-end of the second shell is connected to the tail-end of the elastic stretch band, and the elastic stretch drives the head-end to be detachably connected to the first shell, the head-end shell of the shell; (h) the bending space of the middle part of the second shell corresponds to the shape of the corresponding side of the hand having the index finger; (i) the tail end of the second shell is a bulging hollow shell, and the tail end shell of the first shell is also a bulging hollow shell.
12. The biosignal acquisition device, wherein the one or more biosignal sensors includes a heart rate and/or a GSR signal acquisition device.

List of reference elements from drawings: 100 biosensor system; 110 first end; 111 first bent section; 115 first end distal housing; 119 first concavity; 120 second end; 121 second bent section; 125 second end distal housing; 129 second concavity; 130 connecting portion; 131 main base; 132 main base axis; 133 connecting band; 135 flexible portion; 137 connecting band axis; 138 pivot point; 139 hinge; 140 control unit; 141 control housing; 146 user input; 150 first sensor unit; 155 first sensor; 155a light emitter; 155b light receiver; 156 emitted light; 158 second sensor; 160 second sensor unit; 165 third sensor; 180 hand; 181 purlicue; 185 lateral portion that is opposite to the purlicue; 190 third sensor unit; 195 third sensor; 400 biosensor system; 410 first end; 411 first bent section; 415 first end housing; 419 first concavity; 420 second end; 421 second bent section; 425 second end housing; 429 second concavity; 430 connecting portion; 431 main base; 432 main base axis; 433 connecting band; 434 connection; 435 flexible portion; 437 connecting band axis; 438 pivot point; 439 hinge; 440 control unit; 441 control housing; 445 user interface; 450 first sensor unit; 455 first sensor; 455a light emitter; 455b light receiver; 456 emitted light; 458 second sensor; 460 second sensor unit; 465 third sensor; 480 hand; 481 purlicue; 482 back of hand; 483 palm.

1-20. (canceled)

21. A biosignal acquisition system comprising:
at least a first sensor unit; and
a sensor-positioning structure configured to extend across a hand of a subject, the sensor positioning structure being configured to maintain the first sensor unit at a first position on a palm of a hand of a subject.

22. The biosignal acquisition system according to claim 21, wherein the sensor-positioning structure includes a first end, the first end being configured to conform around and couple to a purlicue of the hand of the subject.

23. The biosignal acquisition system according to claim 22, wherein the first end is C-shaped and includes a first bent section that is rigid or at least semi-rigid, the first bent section defining a first concavity and so being shaped and configured to conform to and couple to the purlicue of a hand of the subject.

24. The biosignal acquisition system according to claim 22, wherein the sensor-positioning structure further includes a second end, the second end being configured to conform around and couple to a lateral portion of the hand that is opposite to the purlicue of the hand of the subject.

25. The biosignal acquisition system according to claim 24, wherein the second end includes a second bent section that is rigid or at least semi-rigid, the second bent section defining a second concavity and so being shaped and configured to conform the lateral portion of the hand that is opposite to the purlicue of the hand of the subject.

26. The biosignal acquisition system according to claim 24, wherein the sensor-positioning structure further includes a connecting portion extending from and coupling to the first end and the second end.

27. The biosignal acquisition system according to claim 26, wherein the connecting portion is configured to extend across the back of the hand of the subject.

28. The biosignal acquisition system according to claim 26, wherein the connecting portion includes a flexible portion having more flexibility than the first end or the second end, the flexible portion providing flex to the connecting portion.

29. The biosignal acquisition system according to claim 21, wherein the first sensor unit includes an oximeter, the oximeter including a light emitter configured to emit light and a light detector configured to detect light emitted by the light emitter that passes through a portion of the hand of the subject,
wherein the sensor-positioning structure is configured to position the light emitter at a position on the back of the hand of the subject and is configured to position the light detector at a position on the palm of the hand of the subject, or
wherein the sensor-positioning structure is configured to position the light emitter at a position on the palm of the hand of the subject and is configured to position the light detector at a position on the back of the hand of the subject.

30. The biosignal acquisition system according to claim 29, wherein the sensor-positioning structure is configured to position the oximeter such that light emitted by the light emitter is transmitted through at least a portion of the purlicue of the hand of the subject.

31. The biosignal acquisition system according to claim 21, wherein the first sensor unit includes galvanic skin response (GSR) sensor.

32. The biosignal acquisition system according to claim 31, wherein the sensor-positioning structure is configured to position the GSR sensor at a position on the palm of the hand.

33. The biosignal acquisition system according to claim 21, wherein the first sensor unit includes an oximeter, the oximeter including a light emitter configured to emit light and a light detector configured to detect light emitted by the light emitter that passes through a portion of the hand of the subject,
wherein the sensor-positioning structure is configured to position the light emitter at a position on the back of the hand of the subject and is configured to position the light detector at a position on the palm of the hand of the subject,
wherein the sensor-positioning structure is configured to position the oximeter such that light emitted by the light emitter is transmitted through at least a portion of the purlicue of the hand of the subject, and
wherein the first sensor unit includes galvanic skin response (GSR) sensor,
wherein the sensor-positioning structure is configured to position the GSR sensor at a position on the palm of the hand.

34. The biosignal acquisition system according to claim 21, wherein the biosignal acquisition system further comprises a control unit arranged in a control housing, the control unit being configured to receive biosignals received from the first sensor unit and transmit data relating to the biosignals received from the first sensor unit to a secondary device that is external to the control unit.

35. The biosignal acquisition system according to claim 34, wherein the control unit is configured to transmit to a smart phone, laptop computer, computer, a server, or another external terminal.

36. The biosignal acquisition system according to claim 34, wherein the control unit is configured to transmit the data relating to the biosignals received from the first sensor unit to the secondary device across a network communication channel, a wireless network, or local area network.

37. The biosignal acquisition system according to claim 26, wherein the connecting portion includes a hinge that provides a pivoting point such that an axis of the first end is arranged at an offset angle (α) from an axis of the second end.

38. The biosignal acquisition system according to claim 21, wherein the sensor-positioning structure includes a first end, the first end defining a first concavity and so being configured to conform around and couple to a purlicue of a hand of the subject,
wherein the sensor-positioning structure further includes a second end, the second end defining a second concavity and so being configured to conform around and couple to a lateral portion of the hand that is opposite to the purlicue of the hand of the subject, and
wherein the biosignal acquisition system further comprises a second sensor unit including a second sensor, the sensor-positioning structure being configured to maintain the second sensor unit at the second end at a second position on the palm of the subject.

39. The biosignal acquisition system according to claim 21, further comprising a third sensor unit including a third sensor,
wherein the sensor-positioning structure is configured to maintain the third sensor unit at a position of the sensor-positioning structure facing away from a back of the hand of the subject.

40. A method of acquisition of data relating to a biosignal, the method comprising:
providing the biosignal acquisition system according to claim 21;
arranging the biosignal acquisition system on a hand of the subject such that the first sensor unit is arranged and maintained at a first position on a palm of a subject;
obtaining biosignals detected by the first sensor unit.
